(19) United States
(12) Patent Application Publication
(10) Pub. No.: US 2008/0213808 A1
(43) Pub. Date: Sep. 4, 2008

(54) MEDIATORS FOR PHOTOMETRIC TESTS AND MEANS AND METHODS RELATING TO USE THEREOF

(76) Inventor: Wolfgang-Reinhold Knappe, Ludwigshafen (DE)

Correspondence Address: ROCHE DIAGNOSTICS OPERATIONS INC., 9115 Hague Road, Indianapolis, IN, 46250-0457, US

(21) Appl. No.: 12/037,962

(22) Filed: Feb. 27, 2008

(30) Foreign Application Priority Data
Feb. 27, 2007 (EP) 07004083.7

(51) Int. Cl. C12Q 1/54; C12Q 1/26; C12Q 1/58; C12Q 1/32

(52) U.S. Cl. 435/11; 435/14; 435/25; 435/26; 435/12

(57) ABSTRACT

Embodiments of a detection reagent, as well as test elements and analytical kits including such reagent, and methods for using such reagent, are disclosed herein for optical detection and/or measurement of an analyte in a fluid sample.

### CLAIM OF PRIORITY

The present application is based on and claims priority to European Patent Application No. 07 004 083.7, filed Feb. 27, 2007, which is hereby incorporated by reference in its entirety.

### TECHNICAL FIELD OF THE INVENTION

The present application relates to optical detection of an analyte in a sample, and more particularly to a detection reagent suitable for this purpose, to methods of using such reagents, and to kits and test elements for use of such reagents.

### BACKGROUND

Measurement systems for biochemical analyses represent important components of clinically relevant analytical methods. The priority in this regard is measurement of analytes which are determined directly or indirectly with the aid of enzymes. The analytes are, in a typical case, converted with the aid of an enzyme-coenzyme complex and then quantified, where appropriate, with use of additional reagents. In this regard the analyte to be determined is brought into contact with a suitable enzyme and a coenzyme, the enzyme mostly being employed in catalytic amounts. The coenzyme is changed by this enzymatic reaction, e.g. oxidized or reduced. This process can be detected directly or indirectly by electrochemical or/and photometric measurement means. Calibration provides a direct relationship of the measurement with the concentration of the analyte to be determined.

However, the analytical methods known from the prior art are associated with certain disadvantages. For example, numerous test strips which allow photometric detection of an analyte employ a detection system which uses glucose-dye oxidoreductase (GilucDOR; EC 1.1.5.2), a PQQ-dependent glucose dehydrogenase, as enzyme. While the analyte substrate is oxidized during the enzymatic conversion, a simultaneous reduction of the corresponding coenzyme takes place. In the case of the coenzyme PQQ (PQQ: pyrroloquinolinequinone; 4,5-dihydro-4,5-dioxo-1H-pyrrolo[2,3-f]quinoline-2,7,9-tricarboxylic acid), reduction takes place to PQQH₂ which in turn can transfer electrons to a reducible optical indicator. The disadvantage of this system is the low specificity of glucose-dye oxidoreductase, which converts not only glucose but also other saccharides such as, for example, maltose and galactose, and thus may provide grossly incorrect results as a result of side reactions.

The NAD⁺-dependent glucose dehydrogenase (EC 1.1.1.47) represents an enzyme which, in the presence of the coenzyme nicotinamide adenine dinucleotide (NAD⁺), catalyzes the oxidation of glucose to glucono-δ-lactone and has a distinctly greater specificity for glucose than glucose-dye oxidoreductase. Thus, in a cuvette test (or in wet-chemical tests or in solution), glucose dehydrogenase converts, besides glucose, only xylose and mannose to the extent respectively of 15% and 8%, whereas galactose and fructose are not substrates of the enzyme (Tietz Textbook of Clinical Chemistry, W.B. Saunders Company, 2nd edition, 1994, editors C. A. Burtis, E. R. Ashwood, pages 964-965).

The NADH formed by reduction of NAD⁺ during the oxidation of glucose by glucose dehydrogenase has only low reactivity and is not converted directly by reducible optical indicators such as, for example, phosphomolybdic acid or 4-nitrosoanilines. In order to counteract this low reactivity of NADH, in practice mediators which increase the reactivity of the coenzyme are employed. Examples of known mediators are diaphorase (EC 1.6.99.2) or the unstable N-methylphenazonium methosulphate.

Phenanthroline quinone compounds have been disclosed for use in amperometric (electrochemical) detection of analytes and biosensors relating to the same. However, electrochemical test elements do not allow an optical check of the plausibility of measured results with color comparison during qualitative or quantitative detection of the analyte.

The object underlying the present invention is therefore to provide devices and methods for detecting analytes in a sample which ensure a simple and cost-effective procedure with, at the same time, high selectivity and measurement reliability.

### SUMMARY

This object and others that will be appreciated by a person of ordinary skill in the art have been achieved according to the embodiments of the present invention disclosed herein. In one embodiment, the present invention comprises a method for the optical detection of an analyte in a sample, comprising the steps:
a) contacting the sample with a detection reagent comprising a nicotinamide-dependent oxidoreductase, a reducible nicotinamide coenzyme, a quinone mediator, and a reducible optical indicator or a reducible optical indicator system,
wherein the analyte is oxidized by the nicotinamide-dependent oxidoreductase, the nicotinamide coenzyme is reduced, and electrons of the reduced nicotinamide coenzyme are transferred by the mediator to the optical indicator or to the optical indicator system, and
b) determining at least one of the presence and the amount of the analyte in the sample by optically detecting the indicator or the indicator system.

The method of the invention is used for optical detection of an analyte in a sample which may originate from any source. In one embodiment, the sample is derived from a body fluid including, but not limited to, whole blood, plasma, serum, lymph, bile, cerebrospinal fluid, urine, and glandular secretions such as, for example, saliva or sweat. In other embodiments, the sample comprises one of whole blood, plasma and serum. In yet other embodiments, the amount of sample necessary to carry out the analysis is typically from about 0.01 μl to about 100 μl, preferably from about 0.1 μl to about 2 μl.

The analyte which is to be determined qualitatively and/or quantitatively can be any biological or chemical substance which can be detected by means of a redox reaction. In one embodiment, the analyte is selected from the group consisting of malic acid, alcohol, ammonium, ascorbic acid, cholesterol, cysteine, glucose, glutathione, glycerol, urea, 3-hydroxybutyrate, lactic acid, 5′-nucleotidase, peptides, pyruvate, salicylate and triglycerides. In other embodiments, the analyte to be determined comprises glucose.

In a further embodiment, the present invention relates to a kit for the optical detection of an analyte in a sample, which includes the detection reagent of the present invention and a carrier, such as a test element. In one embodiment, the test element comprises an application zone for applying the sample, a reaction zone for reacting the analyte with the detection reagent, and a detection zone for determining the presence or/and the amount of analyte in the sample by optically detecting the indicator or the indicator system. In other embodiments, the test element further comprises a waste zone. In embodiments for the kit, reference is made concerning possible configurations of the test element to the statements in the context of the description of the embodiments of the method of the present invention.

In yet a further embodiment, the present invention relates to a test element for the optical detection of an analyte in a sample. In one embodiment, the test element comprises an application zone for applying the sample, a reaction zone which comprises the detection reagent of the invention, comprising a nicotinamide-dependent oxidoreductase, a reducible nicotinamide coenzyme, a quinone mediator, and a reducible optical indicator or a reducible optical indicator system, for reacting the analyte with the detection reagent, a detection zone for determining the presence or/and the amount of the analyte in the sample by optically detecting the indicator or the indicator system, and optionally a waste zone.

Embodiments of the test element of the present invention can be used for example for determining analytes from the group consisting of malic acid, alcohol, ammonium, ascorbic acid, cholesterol, cysteine, glucose, glutathione, glycerol, urea, 3-hydroxybutyrate, lactic acid, 5′-nucleotidase, peptides, pyruvate, salicylate and triglycerides. In one embodiment, a test element according to the present invention serves to determine glucose in whole blood, plasma or serum and includes a detection reagent which comprises glucose dehydrogenase as nicotinamide-dependent oxidoreductase and NAD(P)⁺ as nicotinamide coenzyme.

The invention is to be explained in more detail by the following figures and examples.

### BRIEF DESCRIPTION OF THE DRAWINGS

The following detailed description of the embodiments of the present invention can be best understood when read in conjunction with the following drawings, where like structure is indicated with like reference numerals and in which:

FIG. 1 shows the reflectance of a test element of the invention with N-methyl-1,10-phenanthrolinium-5,6-quinone as mediator as a function of the wavelength before and after contacting with a sample of 10 μl of EDTA-venous blood which contains 400 mg/dl of glucose.

In order that the present invention may be more readily understood, reference is made to the following detailed descriptions and examples, which are intended to illustrate the present invention, but not limit the scope thereof.

### DETAILED DESCRIPTION OF EMBODIMENTS OF THE PRESENT INVENTION

The following descriptions of the embodiments are merely exemplary in nature and are in no way intended to limit the present invention or its application or uses.

In the context of the present invention, a sample for detecting the analyte is brought into contact with a detection reagent which is likewise according to the invention and which includes a nicotinamide-dependent oxidoreductase, a reducible nicotinamide coenzyme, a quinone mediator, and a reducible optical indicator or a reducible optical indicator system, wherein the analyte is oxidized by the nicotinamide-dependent oxidoreductase, the nicotinamide coenzyme is reduced during this, and electrons of the reduced nicotinamide coenzyme are transferred by the mediator to the optical indicator or to the optical indicator system.

The nicotinamide-dependent oxidoreductase is preferably a dehydrogenase, and in particular alcohol dehydrogenase (EC 1.1.1.1), formaldehyde dehydrogenase (EC 1.2.1.46), glucose dehydrogenase (EC 1.1.1.47), glucose-6-phosphate dehydrogenase (EC 1.1.1.49), glycerol dehydrogenase (EC 1.1.1.6), 3-hydroxybutyrate dehydrogenase (EC 1.1.1.30), 3-hydroxysterotid dehydrogenase, e.g., 3α-hydroxysteroid dehydrogenase (EC 1.1.1.209), lactate dehydrogenase (EC 1.1.1.27, EC 1.1.1.28), malate dehydrogenase (EC 1.1.1.37) or amino-acid dehydrogenase, e.g. L-amino-acid dehydrogenase (EC 1.1.3.4). The nicotinamide-dependent oxidoreductase is particularly preferably glucose dehydrogenase.

Embodiments of the present invention provide for the reducible nicotinamide coenzyme to be any natural or synthetic low molecular weight molecule which comprises nicotinamide as a constituent, is reducible and has the ability to form an enzyme-coenzyme complex with a nicotinamide-dependent oxidoreductase as described above. The reducible nicotinamide coenzyme of the present invention is preferably a naturally occurring nicotinamide coenzyme, in particular nicotinamide adenine dinucleotide (NAD⁺) or nicotinamide adenine dinucleotide phosphate (NADP⁺), from which NADH and NADPH, respectively, are produced by reduction. It is, however, possible where appropriate, if this appears expedient, also to employ derivatives of NAD⁺ or NADP⁺. Derivatives of NAD⁺ and NADP⁺ which may be useful in the context of the present invention include inter alia CarbaNAD derivatives and are described for example in WO 98/33936, WO 01/94370, DE 10 2006 035 020.0 and in two publications which appeared in 1989 (Slama et. al., Biochemistry, 1989, 27, 183-193; Slama et al., Biochemistry, 1989, 28, 7688-7694), the disclosure of which is incorporated herein by reference.

Embodiments of the detection reagent which is employed in the context of the method of the present invention comprises, besides the nicotinamide-dependent oxidoreductase and the reducible nicofinamide coenzyme, also a quinone mediator. The mediator may be any quinone which is able to transfer electrons of a reduced nicotinamide coenzyme by redox reactions to an optical indicator or to an optical indicator system. During this electron transfer, the quinone is typically reduced in the first step to a semiquinone or a dihydroquinione, before reoxidation of the reduced form by the optical indicator or by the optical indicator system takes place.

In one embodiment of the method of the present invention, the mediator comprises an o-beizoquinone or a p-benzoquinone, which is optionally fused to at least one further cyclic compound. The term “fused to” as used in the context of the present invention means that the quinone compound and the further cyclic compound(s) each share at least two atoms of their basic cyclic structure. The further cyclic compound(s) may be substituted or unsubstituted, and may, besides carbon and hydrogen, comprise independently of one another one or more heteroatoms such as, for example, nitrogen, oxygen or/and sulphur. Examples of suitable cyclic compounds which can be fused to the o-benzoquinone or p-benzoquinone include in particular aromatic and heteroaromatic ring systems such as benzene, naphthalene, pyridine, pyrimidine, pyrazine, pyridazine, triazine, quinoline and isoquinoline, but are not limited thereto.

In another embodiment, the mediator comprises a phenanthrenequinone, a phenanthrolinequinone or a benzo[h]quinolinequinone. In this context, 1,10-phenanthrolinequinones, 1,7-phenanthrolinequinones, 4,7-phenanthrolinequinones, benzo[h]quinolinequinones, and their N-alkylated or N,N′-dialkylated salts have proved to be suitable. In the case of N-alkylated or N,N′-dialkylated salts of the above compounds, any anion can act as counter ion of the mediator, with preference being given to halides, trifluoromethanesulphonate or other anions which increase the solubility as counter ion. In one embodiment, a halide or trifluoromethanesulphonate is employed as counter ion.

In yet other embodiments, the mediator comprises one of a 1,10-phenanthroline-5,6-quinone of the general formula (I), a 1,7-phenanthroline-5,6-quinone of the general formula (II) and a 4,7-phenanthroline-5,6-quinone of the general formula (III), or a salt or reduced form thereof:

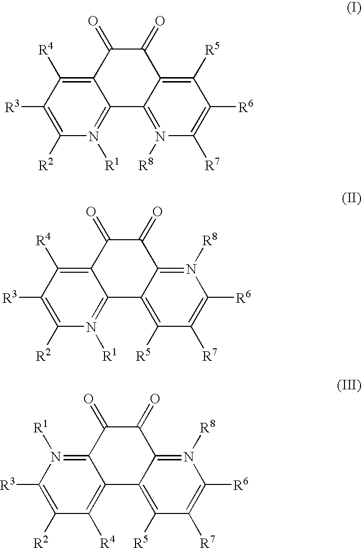

in which:

R² through R⁷ in each case independently denotes H, halogen, OH, O(alkyl), OCO(alkyl), S(alkyl), NH₂, NH(alkyl), N(alkyl)₂, [N(alkyl)₃]⁺, CN, NO₂, COOH, SO₃H, a linear or branched alkyl radical, a cycloalkyl radical, an aryl radical or a heteroaryl radical, which radicals may in each case optionally be substituted at least one time; and

R¹ and R⁸ in each case independently denotes a free electron pair, H, or a linear or branched alkyl radical which may optionally be substituted at least one time.

In one embodiment, one or both of the radicals R¹ and R⁸ comprise a linear or branched alkyl radical which may optionally be substituted at least one time, such as to be a methyl radical, with the counter ion of the mediator being as defined above.

As used herein, the term “halogen” includes fluorine, chlorine, bromine and iodine.

As used herein, the term “alkyl” refers to a linear or branched hydrocarbon radical having 1-30 carbon atoms and a valence bond on any carbon atom of the radical. Alkyl typically comprises a hydrocarbon radical having 1-12 carbon atoms, in several embodiments having 1-6 carbon atoms. In yet other embodiments, the hydrocarbon radicals have 1-4 carbon atoms and include methyl, ethyl, n-propyl, isopropyl, n-butyl, isobutyl, sec-butyl and tert-butyl.

As used, herein, the term “cycloalkyl” refers to a cyclic hydrocarbon radical having 3-20 carbon atoms and a valence bond on any carbon atom of the ring. Cycloalkyl typically comprises a cyclic hydrocarbon radical having 3-12 carbon atoms, in several embodiments having 3-8 carbon atoms. In yet other embodiments, the cyclic hydrocarbon radicals include cyclopropyl, cyclobutyl, cyclopentyl, cyclohexyl, cycloheptyl and cyclooctyl.

As used herein, the term “aryl” refers to an aromatic ring system having 3-20 ring atoms, in several embodiments having 6-14 ring atoms, which, besides carbon, comprises hydrogen and has a valence bond on any carbon atom of the ring. Examples of aryls within the meaning of the present invention include benzene, naphthalene, anthracene and phenanthrene.

As used herein, the term “heteroaryl” refers to an aromatic ring system having 3-20 ring atoms, in several embodiments having 5-14 ring atoms, which, besides carbon and hydrogen, comprises at least one heteroatom and has a valence bond on any carbon atom or on any nitrogen atom of the ring. Examples of heteroaryls having 5 ring atoms include thienyl, thiazolyl, furanyl, pyrrblyl, oxazolyl, imidazolyl, pyrazolyl, isoxazolyl, isothiazolyl, oxadiazolyl, triazolyl, tetrazolyl and thiadiazolyl. Heteroaryls having 6 ring atoms include for example pyridyl, pyridazinyl, pyrimidinyl, pyrazinyl and triazinyl. Heteroaryls having 5 or 6 ring atoms typically have 1-4 nitrogen atoms or/and 1-2 oxygen atoms or/and 1-2 sulphur atoms, which may occur in all subcombinations in the ring system as long as they do not exceed the number fixed for the respective heteroatom and in total the maximum number of four heteroatoms.

As used herein, the expression “optionally substituted one or more times” means that the respective radical may be either unsubstituted or else substituted one or more times, suitable substituents being in particular halogen, OH, O(alkyl), OCO(alkyl), S(alkyl), primary, secondary, tertiary and quaternary amino groups, CN, NO₂ or/and acid groups such as, for example, COOH and SO₃H. The substituents typically comprise groups which increase the solubility of the mediator in the sample to be investigated, such as, for example, quaternary amino groups, COOH and SO₃H.

In a typical embodiment of the detection reagent, the quinone mediator according to the present invention comprises one of N-methyl-1,10-phenanthrolinium-5,6-quinone, N,N′-dimethyl-1,10-phenanthrolinium-5,6-quinone, N-methyl-1,7-phenanthrolinium-5,6-quinone, N,N′-dimethyl-1,7-phenanthrolinium-5,6-quinone, N-methyl-4,7-phenanthrolinium-5,6-quinone, and N,N′-dimethyl-4,7-phenanthrolinium-5,6-quinone, with the counter ion of the mediator being as defined above.

According to the present invention, it is possible to use as an optical indicator or as an optical indicator system any substance which is reducible and, on reduction, undergoes a detectable change in its optical properties such as, for example, color, fluorescence, reflectance, transmission, polarization and/or refractive index. Determination of the presence and/or the amount of the analyte in the sample by optical detection can take place with the naked eye and/or by means of a detection apparatus using a photometric method which appears suitable to the skilled person. In one embodiment of the present invention, heteropoly acids and in particular phosphomolybdic acid are used as optical indicators which are reduced to the corresponding heteropoly blue.

In a further embodiment, the method of the invention is carried out on a carrier which includes an application zone for applying the sample, a reaction zone for reacting the analyte with the detection reagent, a detection zone for determining the presence and/or the amount of the analyte in the sample by optically detecting the indicator or the indicator system, and optionally a waste zone. The carrier may consist of a single capillary active material, or may alternatively be composed of a plurality of capillary active materials which are identical or different. These materials are in close contact with one another so that in this way there is formation of a path which serves to transport liquid and via which a liquid sample proceeds from the application zone through the reaction zone to the detection zone and, where appropriate, to the waste zone. In one embodiment, the carrier comprises a test element such as, for example, a test strip or a biosensor. Test elements which can be used in the context of the present invention are described inter alia in U.S. Pat. No. 5,271,895, U.S. Pat. No. 6,207,000, U.S. Pat. No. 6,540,890, U.S. Pat. No. 6,755,949, U.S. Pat. No. 7,008,799, U.S. Pat. No. 7,025,836, U.S. Pat. No. 7,067,320, US 2003/0031592 A1 and US 2006/0003397 A1, the disclosures of each of which are incorporated herein by reference.

In one embodiment, the sample to be investigated is applied to the application zone of the carrier by for example immersing the application zone of the carrier in the sample, or by placing drops of the sample on the application zone of the carrier. The alternative possibility is for the sample first to be taken up by a dry or moist transfer element from which the sample is then, where appropriate after moistening, applied to the application zone of the carrier. The transfer element is typically a sterile device which may include natural or/and synthetic materials. Suitable transfer elements are described for example in DE 44 39 429 and DE 196 22 503, the disclosures of each of which are incorporated herein by reference.

The reaction zone typically includes a detection reagent according to the present invention and serves to convert the analyte. During this conversion, the analyte is oxidized by the nicotinamide-dependent oxidoreductase, while a simultaneous reduction of the nicotinamide coenzyme takes place. The electrons of the reduced nicotinamide coenzyme are subsequently transferred by the mediator to the optical indicator or to the optical indicator system which undergoes a change of its optical properties. The change of optical properties of the optical indicator or of the optical indicator system is detected in the detection zone of the test element.

In one embodiment, the method of the invention permits rapid determination of the presence and/or the amount of the analyte in the sample to be investigated. The expression “rapid determination” as used in the context of the present application means that the determination of the presence and/or the amount of the analyte takes place within a period of from 1 to 30 seconds after contacting the sample to be investigated with the detection reagent, with a period of from 2 to 15 seconds being an exemplary useful time period.

In order to ensure such short reaction times, in one embodiment the quinone mediator dissolves rapidly in the sample to be investigated, i.e. for example in a period of a few seconds after contacting the sample with the detection reagent. A rapid dissolution of the mediator can be achieved for example by introducing suitable substituents which increase the solubility into the mediator molecule, by encapsulating the mediator in micelles, by a very fine, virtually amorphous distribution of the mediator in a test element, or/and in the case of salts by choosing a suitable counter ion.

According to embodiments of the detection reagent and carrier described above, a further aspect of the present invention includes a kit for the optical detection of an analyte in a sample, which includes the detection reagent of the present invention and a carrier, such as a test element. In one embodiment, the test element comprises an application zone for applying the sample, a reaction zone for reacting the analyte with the detection reagent, and a detection zone for determining the presence or/and the amount of analyte in the sample by optically detecting the indicator or the indicator system. In other embodiments, the test element further comprises a waste zone. In embodiments for the kit, reference is made concerning possible configurations of the test element to the statements in the context of the description of the embodiments of the method of the present invention.

According to embodiments of the detection reagent and carrier described above, a further aspect of the present invention includes a test element for the optical detection of an analyte in a sample. In one embodiment, the test element comprises an application zone for applying the sample, a reaction zone which comprises the detection reagent of the invention, comprising a nicotinamide-dependent oxidoreductase, a reducible nicotinamide coenzyme, a quinone mediator, and a reducible optical indicator or a reducible optical indicator system, for reacting the analyte with the detection reagent, a detection zone for determining the presence or/and the amount of the analyte in the sample by optically detecting the indicator or the indicator system, and optionally a waste zone.

### EXAMPLE

The foregoing aspects of the present invention may be more clearly understood in view of the following example of manufacturing a test element.

Test elements according to certain embodiments of the present invention were produced by first applying a 5 mm-wide double-sided adhesive tape (polyester backing and synthetic rubber adhesive) to a tapelike, 50 mm-wide titanium dioxide-containing polyester support layer parallel to and at a distance of 18.6 mm from (measured from the left-hand edge of the adhesive tape) its left-hand edge. Two holes, a positioning hole and an inspection and measuring hole, were cut out of this composite in each case at a distance of 6 mm, the centers of which were located on a line running perpendicular to the long axis of the carrier strip. The first hole, the positioning hole, was circular and had a diameter of 2.6 mm, and the distance of the centre of the hole from the left-hand edge of the carrier strip was 4 mm. The second hole was likewise round with a diameter of 4 mm. The distance of the centre of the second hole from the left-hand edge of the carrier strip was 21 mm. The protective paper of the double-sided adhesive tape was then stripped off.

To produce a detection layer assembled from 2 film layers, the following components were combined as pure substances or in the form of stock solutions in the following composition, and mixed by stirring, in a glass beaker:

| | |
|---|---|
| Water: | 60.5 g |
| Xanthan gum: | 0.34 g |
| 0.1 M phosphate buffer pH 6.5: | 20.0 g |
| Tetraethylammonium chloride: | 0.14 g |
| N-Octanoyl-N-methylglucamide: | 0.17 g |
| Sodium N-methyl-N-oleoyltaurate: | 0.03 g |
| Polyvinylpyrrolidone (MW 25 000): | 0.87 g |
| Transpafill (sodium aluminum silicate): | 5.45 g |
| Polyvinyl propionate dispersion | 4.88 g |
| (50% by weight in water): | |
| N-Methyl-1,10-phenanthrolinium-5,6- | 0.11 g |
| quinone trifluoromethanesulphonate: | |
| Sodium chloride: | 0.98 g |
| NAD: | 0.99 g |
| Hexasodium 2,18-phosphomolybdate: | 0.64 g |
| Glucose dehydrogenase: | 327 KU (1.35 g) |
| Potassium hexacyanoferrate(III): | 0.02 g |
| 1-Hexanol: | 0.17 g |
| 1-Methoxy-2-propanol: | 4.30 g |

The total mass was adjusted to a pH of 6.7 with NaOH and then applied with a weight per unit area of 89 g/m² to a 125 μm-thick polycarbonate sheet, and dried.

Subsequently, the following components were combined as pure substances or in the form of stock solutions in the following composition, and mixed by stirring, in a glass beaker:

| | |
|---|---|
| Water: | 65.0 g |
| Gantrez (methyl vinyl ether-maleic acid copolymer): | 1.36 g |
| NaOH: | 0.30 g |
| 0.1 M phosphate buffer pH 6.5: | 4.41 g |
| Tetraethylammonium chloride: | 0.34 g |
| N-Octanoyl-N-methylglucamide: | 0.34 g |
| Sodium N-methyl-N-oleoyltaurate: | 0.03 g |
| Polyvinylpyrrolidone (MW 25 000): | 1.86 g |
| Titanium dioxide E 1171: | 14.37 g |
| Precipitated silica 320: | 1.96 g |
| Polyvinyl propionate dispersion (50% by weight in water): | 5.77 g |
| N-Methyl-1,10-phenanthrolinium-5,6- | 0.38 g |
| quinone trifluoromethanesulphonate: | |
| Hexasodium 2,18-phosphomolybdate: | 1.11 g |
| Potassium hexacyanoferrate(III): | 0.01 g |
| 1-Hexanol: | 0.16 g |
| 1-Methoxy-2-propanol: | 4.26 g |

The total mass was adjusted to a pH of approx. 6.7 with NaOH and applied as second layer with a weight per unit area of 104 g/m² to the monocoated polycarbonate sheet described above, and dried.

A 5 mm-wide strip of the detection layer produced in this way was stuck with accurate fit by the sheet side to the perforated double-sided adhesive tape located on the backing layer. Directly adjacent to the detection layer, double-sided adhesive tapes were stuck on both sides of the carrier sheet as spacers, with one spacer being 6 mm, and the second spacer being 9 mm, wide in the present case. The protective sheet of the two double-sided adhesive tapes was then stripped off.

A 20 mm-wide, strip of a spreading fabric as described for example in EP 0 995 994 A2, the disclosure of which is incorporated herein by reference, was placed on this composite and bonded by pressing. Subsequently, two one-sided adhesive tapes were stuck as coverings on the spreading fabric in such a way that the spacers were completely covered and at least a slight overlap with the reactive region was also present. The tape product was cut into 6 mm-wide test elements, with the measuring hole being in the centre of the test element.

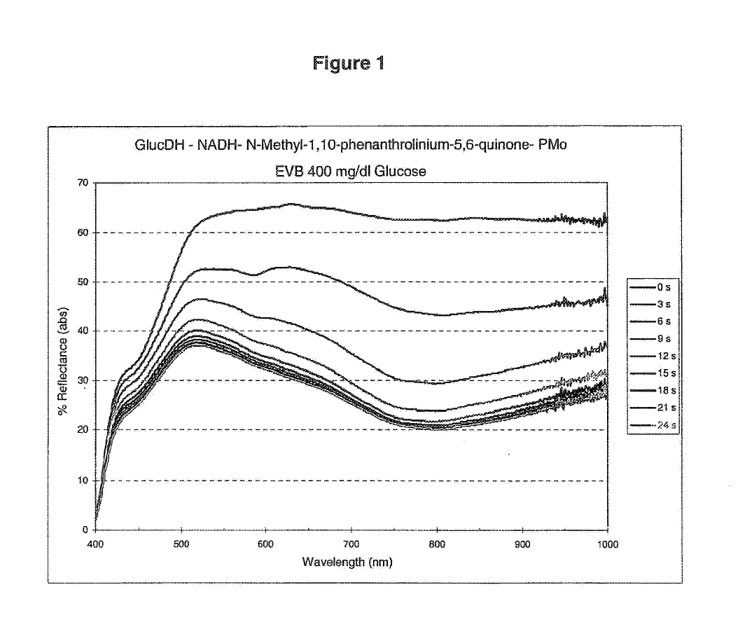

According to the reflectance measurements of a test element of the present invention shown in FIG. 1, the spectra demonstrate a rapid reaction, which is substantially complete after 12 seconds. The spectra were recorded at an interval of 3 s in each case by means of a “TIDAS 16” reflectance spectrometer which consisted of a CLH tungsten-halogen light source, a reflectance measuring head and an MCS diode array simultaneous spectrometer (each from Zeiss), which were in turn connected by light guides.

The features disclosed in the above description, the claims and the drawing may be important both individually and in any combination with one another for implementing the invention in its various embodiments.

It is noted that terms like “preferably”, “commonly”, and “typically” are not utilized herein to limit the scope of the claimed invention or to imply that certain features are critical, essential, or even important to the structure or function of the claimed invention. Rather, these terms are merely intended to highlight alternative or additional features that may or may not be utilized in a particular embodiment of the present invention.

For the purposes of describing and defining the present invention it is noted that the term “substantially” is utilized herein to represent the inherent degree of uncertainty that may be attributed to any quantitative comparison, value, measurement or other representation. The term “substantially” is also utilized herein to represent the degree by which a quantitative representation may vary from a stated reference without resulting in a change in the basic function of the subject matter at issue.

Having described the present invention in detail and by reference to specific embodiments thereof, it will be apparent that modification and variations are possible without departing from the scope of the present invention defined in the appended claims. More specifically, although some aspects of the present invention are identified herein as preferred or particularly advantageous, it is contemplated that the present invention is not necessarily limited to these preferred aspects of the present invention.

What is claimed is:

1. A method for optical detection of an analyte in a sample, comprising the steps:
(a) contacting the sample with a detection reagent comprising a nicotinamide-dependent oxidoreductase, a reducible nicotinamide coenzyme, a quinone-based mediator, and a reducible optical indicator or a reducible optical indicator system, wherein upon or after said contacting the analyte is oxidized by the nicotinamide-dependent oxidoreductase, the nicotinamide coenzyme is reduced, and electrons of the reduced nicotinamide coenzyme are transferred by the quinone mediator to the indicator or to the indicator system; and
(b) determining at least one of the presence and the amount of the analyte in the sample by optically detecting the indicator or the indicator system.

2. The method according to claim 1, wherein the sample comprises a body fluid.

3. The method according to claim 1, wherein the body fluid comprises one of the group consisting of whole blood, plasma and serum.

4. The method according to claim 1, wherein the analyte comprises a compound selected from the group consisting of malic acid, alcohol, ammonium, ascorbic acid, cholesterol, cysteine, glucose, glutathione, glycerol, urea, 3-hydroxybutyrate, lactic acid, 5′-nucleotidase, peptides, pyruvate, salicylate and triglycerides.

5. The method according to claim 4, wherein the analyte is glucose.

6. The method according to claim 1, wherein the nicotinamide-dependent oxidoreductase comprises a dehydrogenase.

7. The method according to claim 6, wherein the dehyrogenase comprises an enzyme selected from the group consisting of alcohol dehydrogenase, formaldehyde dehydrogenase, glucose dehydrogenase, glucose-6-phosphate dehydrogenase, 3-hydroxybutyrate dehydrogenase, 3-hydroxysteroid dehydrogenase, lactate dehydrogenase, malate dehydrogenase, and amino-acid dehydrogenase.

8. The method according to claim 7, wherein the dehydrogenase comprises glucose dehydrogenase.

9. The method according to claim 1, wherein the nicotinamide coenzyme comprises NAD(P)⁺.

10. The method according to claim 1, wherein the mediator comprises one of an o-benzo-quinione and a p-benzoquinone.

11. The method according to claim 10, wherein the o- or p-benzoquinone further comprises at least one cyclic compound fused thereto.

12. The method according to claim 1, wherein the mediator comprises a compound selected from the group consisting of 1,10-phenanthrolinequinone, 1,7-phenanthrolinequinone, 4,7-phenanthrolinequinone, benzo[h]quinolinequinone, and N-alkylated or N,N′-dialkylated salts thereof.

13. The method according to claim 12, wherein the mediator comprises 1,10-phenanthrolinequinone of the general formula (I) or a salt or a reduced form thereof: 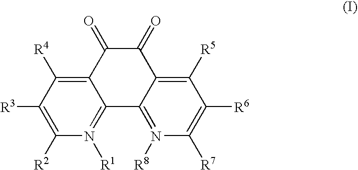
in which:
R² through R⁷ in each case independently denotes H, halogen, OH, O(alkyl), OCO(alkyl), S(alkyl), NH₂, NH(alkyl), N(alkyl)₂, [N(alkyl)₃]⁺, CN, NO₂, COOH, SO₃H, a linear or branched alkyl radical, a cycloalkyl radical, an aryl radical or a heteroaryl radical, which radicals may in each case optionally be substituted at least one time; and
R¹ and R⁸ in each case independently denotes a free electron pair, H, or a linear or branched alkyl radical which may optionally be substituted one or more times.

14. The method according to claim 12, wherein the mediator comprises 1,7-phenanthrolinequinone of the general formula (II) or a salt or a reduced form thereof: 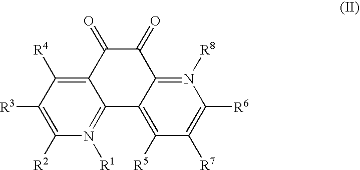
in which:
R² through R⁷ in each case independently denotes H, halogen, OH, O(alkyl), OCO(alkyl), S(alkyl), NH₂, NH(alkyl), N(alkyl)₂, [N(alkyl)₃]⁺, CN, NO₂, COOH, SO₃H, a linear or branched alkyl radical, a cycloalkyl radical, an aryl radical or a heteroaryl radical, which radicals may in each case optionally be substituted at least one time; and
R¹ and R⁸ in each case independently denotes a free electron pair, H, or a linear or branched alkyl radical which may optionally be substituted one or more times.

15. The method according to claim 12, wherein the mediator comprises 4,7-phenanthrolinequinone of the general formula (III) or a salt or a reduced form thereof: 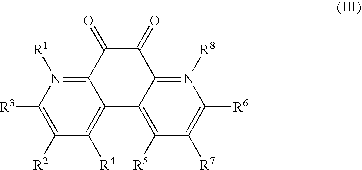
in which:
R² through R⁷ in each case independently denotes H, halogen, OH, O(alkyl), OCO(alkyl), S(alkyl), NH₂, NH(alkyl), N(alkyl)₂, [N(alkyl)₃]⁺, CN, NO₂, COOH, SO₃H, a linear or branched alkyl radical, a cycloalkyl radical, an aryl radical or a heteroaryl radical, which radicals may in each case optionally be substituted at least one time; and,
R¹ and R⁸ in each case independently denotes a free electron pair, H, or a linear or branched alkyl radical which may optionally be substituted one or more times.

16. The method according to claim 12, wherein the mediator comprises a compound selected from the group consisting of N-methyl-1,10-phenanthrolinium-5,6-quinone, N,N′-dimethyl-1,10-phenanthrolinium-5,6-quinone, N-methyl-1,7-phenanthrolinium-5,6-quinone, N,N′-dimethyl-1,7-phenanthrolinium-5,6-quinone, N-methyl-4,7-phenanthrolinium-5,6-quinone or N,N′-dimethyl-4,7-phenanthrolinium-5,6-quinone.

17. The method according to claim 1, wherein the detection reagent comprises an optical indicator comprising phosphomolybdic acid.

18. The method according to claim 1, wherein the method is carried out using a carrier, the carrier comprising:
(a) an application zone for applying the sample;
(b) a reaction zone for reacting the analyte with the detection reagent; and
(c) a detection zone for determining at least one of the presence and the amount of the analyte in the sample by optically detecting the indicator or the indicator system.

19. The method according to claim 18, wherein the sample is applied directly to the application zone.

20. The method according to claim 18, wherein the sample is taken up by means of a transfer element and then, where appropriate after moistening, applied to the application zone.

21. The method according to claim 18, wherein the carrier comprises a test element.

22. A detection reagent for optical detection of an analyte in a sample, comprising
(a) a nicotinamide-dependent oxidoreductase;
(b) a reducible nicotinamide coenzyme;
(c) a quinone mediator; and
(d) a reducible optical indicator or a reducible optical indicator system.

23. A kit comprising a detection reagent according to claim 22 and a test element for the optical detection of an analyte in a sample, wherein the test element comprises
(a) an application zone for applying the sample;
(b) a reaction zone for reacting the analyte with the detection reagent; and
(c) a detection zone for determining the presence or/and the amount of the analyte in the sample by optically detecting the indicator or the indicator system.

24. A test element for optical determination of an analyte in a sample, comprising
(a) an application zone for applying the sample;
(b) a reaction zone including a detection reagent comprising a nicotinamide-dependent oxidoreductase, a reducible nicotinamide coenzyme, a quinone mediator, and a reducible optical indicator or a reducible optical indicator system, configured for reacting the analyte with the detection reagent;
(c) a detection zone for determining at least one of the presence and the amount of the analyte in the sample by optically detecting the indicator or the indicator system; and
(d) a waste zone.
